(19) United States
(12) Patent Application Publication
(10) Pub. No.: US 2026/0272883 A1
(43) Pub. Date: Sep. 17, 2026

(54) SOLID STATE FORMS OF PIRTOBRUTINIB AND PROCESS FOR THE PREPARATION OF INTERMEDIATE THEREOF

(71) Applicant: DR. REDDY'S LABORATORIES LIMITED, Hyderabad (IN)

(72) Inventors: Rajeev Rehani Budhdev, Hyderabad (IN); Srinivas Oruganti, Hyderabad (IN); Saikat Sen, Kolkata (IN); Rao Pallavi, Hyderabad (IN); Sharmistha Pal Datta, Bangalore (IN); Mohan Kumar Kottur, Hyderabad (IN); Mohammad Sirazoddin, Jharasangam, Sangaredy dist. (IN)

(73) Assignee: DR. REDDY'S LABORATORIES LIMITED, Hyderabad (IN)

(21) Appl. No.: 19/166,675

(22) PCT Filed: Mar. 19, 2024
(86) PCT No.: PCT/IN2024/050281

§ 371 Date: Sep. 18, 2025

(30) Foreign Application Priority Data
Mar. 20, 2023 (IN) 202341018878
May 9, 2023 (IN) 202341032720
Jun. 8, 2023 (IN) 202341039408
Aug. 31, 2023 (IN) 202341058356

(51) Int. Cl. A61K 31/415; C07D 231/38

(52) U.S. Cl. CPC A61K 31/415; C07D 231/38; C07B 2200/13

(57) ABSTRACT

Aspects of the present application relates to solid state forms of Pirtobrutinib such as novel crystalline form P1, amorphous solid dispersions comprising Pirtobrutinib and pharmaceutically acceptable carriers and processes thereof, wherein the pharmaceutically acceptable carrier is selected from the group consisting of HPMC phthalate, HPC, Eudragit L 100, microcrystalline cellulose, carboxymethyl cellulose, mannitol, Neusilin, cellulose acetate phthalate, polyvinyl alcohol, polylactic-co-glycolic acid, polyvinyl acetate phthalate, sodium alginate, beta cyclodextrin, and mixtures thereof. Aspects of the present application relates to process for the preparation of [(1S)-2,2,2-trifluoro-1-methyl-ethyl]hydrazine or its salts.

### CROSS REFERENCE

This application claims the priority of our Indian provisional applications 202341018878 filed on 20 Mar. 2023, 202341032720 filed on 9 May 2023, 202341039408 filed on 8 Jun. 2023, 202341058356 filed on 31 Aug. 2023.

### FIELD OF THE INVENTION

The present application is related to solid state forms of Pirtobrutinib, specifically crystalline form P1 of Pirtobrutinib and amorphous solid dispersion of Pirtobrutinib and processes for the preparation of intermediate thereof.

### BACKGROUND OF THE INVENTION

The drug compound having the adopted name “Pirtobrutinib” has chemical name: (S)-5-amino-3-(4-((5-fluoro-2-methoxybenzamido)methyl) phenyl)-1-(1,1,1-trifluoropropan-2-yl)-1H-pyrazole-4-carboxamide of formula (I) and its chemical structure is as below:

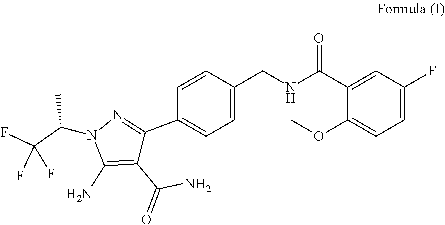 [SMILES: COc1ccc(F)cc1C(=O)NCc1ccc(-c2nn([C@@H](C)C(F)(F)F)c(N)c2C(N)=O)cc1]

Pirtobrutinib is an orally available, small molecule ATP-competitive inhibitor of BTK. Pirtobrutinib is approved by USFDA under the trade name of Jaypicra® for the treatment of adult patients with relapsed or refractory mantle cell lymphoma (MCL).

U.S. Pat. No. 10,342,780 B2 discloses Pirtobrutinib and a process for preparation thereof. The process involves preparation of racemic[(2,2,2-trifluoro-1-methyl-ethyl]hydrazine, which is a key intermediate, and its further use in the preparation of Pirtobrutinib. Since, said racemic intermediate has been used for preparation, the process produces a racemic Pirtobrutinib, which is then subjected to preparative SFC to produce the (S)-enantiomer of Pirtobrutinib, the desired isomer for Pirtobrutinib API. Isolation of the required isomer from the racemate at the last stage leads to material wastage and huge erosion in the yield of Pirtobrutinib. In addition, this process is not viable at industrial scale owing to the involvement of chiral chromatography.

PCT publication no. WO 2022/056100 A1 discloses a process for the preparation of [(1S)-2,2,2-trifluoro-1-methyl-ethyl]hydrazine by reducing 2-phenyl-N-(1,1,1-trifluoropropan-2-ylidene)acetohydrazide with sodium borohydride or hydrogenation using a palladium or platinum catalyst.

However, none of these reducing agents are viable for green process for preparation of Pirtobrutinib. Also, palladium and platinum are heavy metals and hence preferable to avoid for the synthesis of an API.

Hence, there remains a need for (S)-selective asymmetric reduction of 2-phenyl-N-(1,1,1-trifluoropropan-2-ylidene)acetohydrazide which in turn can afford the required (S)-enantiomer of [(2,2,2-trifluoro-1-methyl-ethyl]hydrazine, which may be used for preparation of (S)-isomer of Pirtobrutinib.

US patent publication number US 2021/0330643 A1 discloses amorphous solid dispersion of Pirtobrutinib HPMC-AS, HPMC, vinylpyrrolidone-vinyl acetate copolymer, and a polyvinylpyrrolidone (PVP), and an excipient. The application further discloses crystalline forms A, B and C of Pirtobrutinib and processes for preparation thereof.

However, there remains a need for alternate solid forms of Pirtobrutinib and preparative processes thereof, exhibiting desired properties such as solubility, bioavailability and stability. Hence, it is desirable to provide a viable and alternate solid form of Pirtobrutinib.

Different forms of Active pharmaceutical ingredients (API) in pharmaceutical compositions can be prepared. In the synthesis of a chemical compound intended for pharmaceutical use, it is necessary to isolate and purify the compound at the completion of the synthetic process and prior to further processing to provide the compound in a pharmaceutical formulation. The isolation and the purification steps, which can be combined or separate consecutive steps, provide the compound as a purified solid with minimal loss of yield during isolation from other components of the reaction mixture and/or during purification to remove impurities from the isolated compound sample. It is desirable to prepare physically and chemically stable crystalline form using regulatory acceptable solvents.

Although methods and materials similar or equivalent to those described herein can be used in the practice or testing of the present application, suitable methods, and materials are described below. All publications, patent applications, patents, and other references mentioned herein are incorporated by reference in their entirety. In addition, the materials, methods, and examples are illustrative only and not intended to be limiting.

### BRIEF DESCRIPTION OF THE DRAWING

FIG. 1 is an illustrative X-ray powder diffraction of crystalline Form P1 of Pirtobrutinib prepared by the method of Example-1.

FIG. 2 is an illustrative X-ray powder diffraction of amorphous form of Pirtobrutinib prepared by the method of Example-2.

FIG. 3 is an illustrative X-ray powder diffraction pattern of amorphous solid dispersion of Pirtobrutinib with HPMC phthalate (1:1, w/w) prepared by the method of Example-3.

FIG. 4 is an illustrative X-ray powder diffraction of amorphous solid dispersion of Pirtobrutinib with HPC (1:1, w/w) prepared by the method of Example-4.

FIG. 5 is an illustrative X-ray powder diffraction of amorphous solid dispersion of Pirtobrutinib with mixture of HPMC phthalate and HPC (1:0.5:0.5, w/w) prepared by the method of Example-5 FIG. 6 is an illustrative X-ray powder diffraction of amorphous solid dispersion of Pirtobrutinib with Eudragit L100 (1:1, w/w) prepared by the method of Example-6.

FIG. 7 is an illustrative X-ray powder diffraction (XRPD) pattern of amorphous solid dispersion of Pirtobrutinib with microcrystalline cellulose (MCC) (1:1, w/w) prepared by the method of example-7.

FIG. 8 is an illustrative XRPD pattern of amorphous solid dispersion of Pirtobrutinib with carboxymethyl cellulose (1:1, w/w) prepared by the method of Example-8.

FIG. 9 is an illustrative XRPD pattern of amorphous solid dispersion of Pirtobrutinib with mannitol (1:1, w/w) prepared by the method of Example-9.

FIG. 10 is an illustrative XRPD pattern of amorphous solid dispersion of Pirtobrutinib with Neusilin (1:1, w/w) prepared by the method of Example-10.

FIG. 11 is an illustrative XRPD pattern of amorphous solid dispersion of Pirtobrutinib with cellulose acetate phthalate (1:1, w/w) prepared by the method of Example-11.

FIG. 12 is an illustrative XRPD pattern of amorphous solid dispersion of Pirtobrutinib with polyvinyl alcohol (1:1, w/w) prepared by the method of Example-12.

FIG. 13 is an illustrative XRPD pattern of amorphous solid dispersion of Pirtobrutinib with polylactic-co-glycolic acid (PLGA) (1:1, w/w) prepared by the method of Example-13.

FIG. 14 is an illustrative XRPD pattern of amorphous solid dispersion of Pirtobrutinib with polyvinyl acetate phthalate (PVAP) (1:1, w/w) prepared by the method of Example-14.

FIG. 15 is an illustrative XRPD pattern of amorphous solid dispersion of Pirtobrutinib with sodium alginate (1:1, w/w) prepared by the method of Example-15.

FIG. 16 is an illustrative XRPD pattern of amorphous solid dispersion of Pirtobrutinib with beta cyclodextrin (1:1, w/w) prepared by the method of Example-16.

FIG. 17 illustrates PXRD pattern of Pirtobrutinib crystalline form P1.

FIG. 18 illustrates Differential Scanning Calorimetry (DSC) of Pirtobrutinib crystalline form P1.

FIG. 19 illustrates Thermogravimetric analysis (TGA) of Pirtobrutinib crystalline form P1.

### SUMMARY OF THE INVENTION

First aspect of the present application is related to a crystalline form P1 of Pirtobrutinib and process for preparation thereof. The crystalline form P1 of Pirtobrutinib may be characterized by a Powdered X-Ray Diffraction (PXRD) pattern having peaks at about 5.80, 6.87, 9.16, 10.74, 11.43, 13.00, 13.78, 14.54±0.2° of 2θ.

Second aspect of the present application is related to amorphous solid dispersion comprising Pirtobrutinib and one more pharmaceutically acceptable carrier and processes for preparation thereof, wherein the pharmaceutically acceptable carrier is selected from a group of HPMC phthalate, HPC, Eudragit L100, microcrystalline cellulose, carboxymethyl cellulose, mannitol, Neusilin, cellulose acetate phthalate, polyvinyl alcohol, polylactic-co-glycolic acid, polyvinyl acetate phthalate, sodium alginate, beta cyclodextrin, and mixtures thereof.

Third aspect of the present application is related to a process for the preparation of [(1S)-2,2,2-trifluoro-1-methyl-ethyl]hydrazine or salts thereof.

Fourth aspect of the present application provides a process for the preparation of Pirtobrutinib, comprising,
a) (S)-selective asymmetric reductive amination of formula (III) to produce a compound of formula (II);

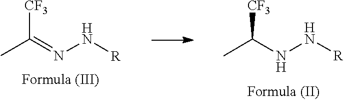

wherein, R is a hydrogen or an amine protecting group
b) converting the compound of formula (II) to Pirtobrutinib of formula (I).

### DETAILED DESCRIPTION

As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values.

Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

The term “about” when used in the present application preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

As used herein, “R” represents hydrogen or an amino-protecting group. The term “N-protecting group” or “amino-protecting group” as used herein refers to those groups intended to protect a nitrogen atom against undesirable reactions during synthetic procedures. N-protecting group includes, carbamates aryloxycarbonyl such as benzoyl, benzyloxycarbonyl (Cbz), fluorenyl methoxy-carbonyl (Fmoc); methyloxycarbonyl, propoxycarbonyl, tert-butyloxycarbonyl (Boc); acyl such as acetyl, propanoyl, iso-butyryl, tert-butyryl, t-butylacetyl, pivaloyl; aroyl groups such as benzoyl; silyl such as trimethylsilyl, tert-butyldimethylsilyl and the like. A thorough discussion of amino-protecting groups have been disclosed in Protective Groups in Organic Synthesis, Fourth edition, Wiley, New York 2006 by T. W. Greene and P. G. M. Wuts, which is incorporated herein by reference.

In the first aspect, the present invention provides a crystalline form P1 of Pirtobrutinib, characterized by a Powdered X-Ray Diffraction (PXRD) pattern having peaks at about 5.80, 6.87, 9.16, 10.74, 11.43, 13.00, 13.78, 14.54±0.2° of 2θ.

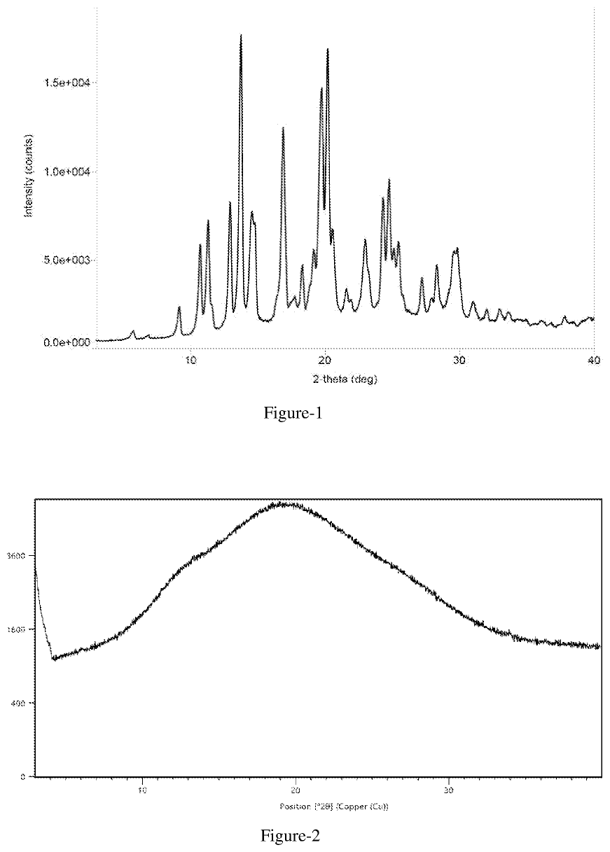

In another embodiment, the crystalline form P1 of Pirtobrutinib, characterized by a Powdered X-Ray Diffraction (PXRD) pattern having the peaks at about 5.80, 6.87, 9.16, 10.74, 13.78, 14.54, 14.82, 17.52, 17.75, 33.62 and 39.21±0.2° of 2θ. In another embodiment, the application provides a crystalline form P1 of Pirtobrutinib, characterized by a PXRD pattern having additional peaks at about 11.32, 11.64, 12.96, 16.86, 17.04, 17.52, 17.75, 18.32, 18.83, 19.19, 19.58, 19.80, 20.23, 20.55, 20.75, 21.59, 21.94, 23.07, 23.30, 24.37, 24.81, 25.13, 25.50, 25.84, 27.25, 27.92, 28.43, 29.88, 31.12, 32.00, 33.04, 34.34, 34.99, 36.17, 36.83, 37.85 and 38.38±0.2° of 2θ. In another embodiment, the application provides a crystalline form P1 of Pirtobrutinib, characterized by a PXRD pattern as illustrated in FIG. 1.

In one embodiment, the application provides a crystalline form P1 of Pirtobrutinib, characterized by Differential Scanning Calorimetry (DSC) having endotherm onset at about 163±5° C. In another embodiment, the application provides a crystalline form P1 of Pirtobrutinib, characterized by Differential Scanning Calorimetry (DSC) as illustrated in FIG. 18.

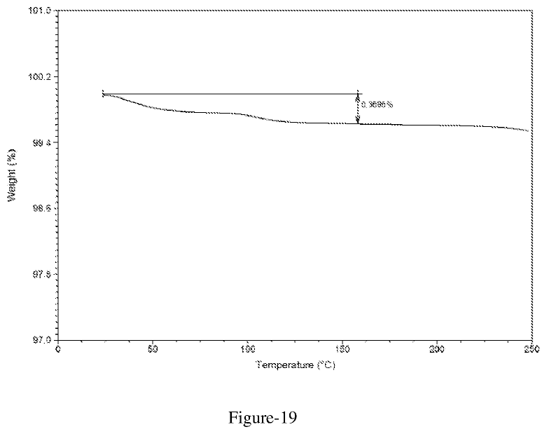

In one embodiment, the application provides a crystalline form P1 of Pirtobrutinib, characterized by Thermal Gravimetric Analysis (TGA), as illustrated in FIG. 19.

In another embodiment, the present invention provides a process for preparation of crystalline form P1 of Pirtobrutinib, comprising:
a) providing a solution of Pirtobrutinib in a solvent;
b) optionally, heating the mixture obtained in step a);
c) combining the mixture obtained in step-a) or step-b) with anti-solvent;
d) isolating the crystalline form P1 of Pirtobrutinib.

The solvent used in step-a) include but are not limited to ester solvents such as ethyl acetate, methyl acetate, propyl acetate or the like; alcohol solvents such as methanol, ethanol, 1-propanol, isopropyl alcohol, n-butanol, tertiary butanol or the like; nitrile solvents such as acetonitrile, propionitrile or the like; ketone solvents such as acetone, 2-butanone or the like; ether solvents such as tetrahydrofuran, methyl tert-butyl ether or the like. In one specific embodiment, the solvent is an ester solvents. Specifically, the solvent is ethyl acetate. In another specific embodiment, the solvent is a ketone solvent. Specifically, the solvent is acetone.

Embodiments of step-a) involves the solution may optionally be treated with carbon, flux-calcined diatomaceous earth (Hy-flow), or any other suitable material to remove color and/or to clarify the solution.

The above step-a) may be carried out in between about 10° C. and about 100° C.

Optionally, the solution obtained above may be filtered to remove any insoluble particles. The insoluble particles may be removed suitably by filtration, centrifugation, decantation, or any other suitable techniques. The solution may be filtered by passing through paper, glass fiber, or other membrane material, or a bed of a clarifying agent such as celite or hy-flow. Depending upon the equipment used and the concentration and temperature of the solution, the filtration apparatus may need to be preheated to avoid premature crystallization.

Embodiments of step-c) involves adding an anti-solvent to the solution obtained in step-a) or step-b), or adding the solution obtained in step-a) or step-b) to the anti-solvent. The reaction mass may be maintained from 15 minutes to 24 hours in embodiments of step-c).

Anti-solvent used in step-c) include but are not limited to alipahtic hydrocarbon solvents such as n-hexane, n-heptane, cyclohexane and the like; aromatic hydrocarbon solvent such as toluene, xylene and the like. Specifically, the anti-solvent is n-hexane. In yet another specific embodiment, the anti-solvent is n-heptane.

The isolation of step-d) can be effected, if desired, by any suitable separation methods such as precipitation, filtration, centrifugation, extraction, acid-base treatment, by scraping, or by shaking the container conventional isolation and refining means such as concentration, concentration under reduced pressure or by a combination of these procedures.

The isolated material may be dried. Drying may suitably be carried out by using any of an air tray dryer, vacuum tray dryer, fluidized bed dryer, spin flash dryer, flash dryer, and the like. The drying may be carried out at atmospheric pressure or above, or under reduced pressures, specifically at temperatures less than about 80° C. and more specifically less than about 60° C. The drying may be carried out for any time period required for obtaining a desired product quality, such as from about 30 minutes to about 24 hours, or longer.

The crystalline form P1 of Pirtobrutinib of the present application may be an anhydrous form. The crystalline form P1 of Pirtobrutinib of the present invention is stable under high humidity and illumination conditions. The crystalline form P1 of Pirtobrutinib is stable for 3 months at accelerated conditions.

In another embodiment, the present application provides pharmaceutical composition comprising crystalline form P1 of Pirtobrutinib with one or more of pharmaceutically acceptable excipients.

The crystalline form P1, obtained by the present application may be advantageously used for providing a pharmaceutical composition comprising Pirtobrutinib. Moreover, the crystalline form P1 may be advantageously used as an input material for preparing an amorphous solid dispersion comprising Pirtobrutinib.

In the second aspect, the present application provides amorphous solid dispersions comprising Pirtobrutinib and one or more pharmaceutically acceptable carrier, wherein the pharmaceutically acceptable carrier is selected from a group of HPMC phthalate, HPC, Eudragit L100, microcrystalline cellulose, carboxymethyl cellulose, mannitol, Neusilin, cellulose acetate phthalate, polyvinyl alcohol, polylactic-co-glycolic acid, polyvinyl acetate phthalate, sodium alginate, beta cyclodextrin, and mixtures thereof.

In embodiments, the present application provides a process for the preparation of amorphous solid dispersion of Pirtobrutinib, comprising the steps of providing a solution of Pirtobrutinib and the pharmaceutically acceptable carrier in a solvent and removing the solvent.

In embodiments, solvent may be selected from the group consisting of methanol, ethanol, 2-propanol, 1-butanol, 2-butanol, 1-pentanol, 2-pentanol, 3-pentanol or mixtures thereof. Preferably, the solvent is methanol.

In embodiments, a solution of Pirtobrutinib and pharmaceutically acceptable carrier may be prepared at any suitable temperatures, such as about 0° C. to about the reflux temperature of the solvent used. Stirring and heating may be used to reduce the time required for the dissolution process.

In embodiments, a solution of Pirtobrutinib and pharmaceutically acceptable carrier may be filtered to make it clear and free of unwanted particles.

In embodiments, the obtained solution may be optionally treated with an adsorbent material, such as carbon and/or hydrose, to remove colored components, etc., before filtration.

In an embodiment, removal of solvent may be carried out by methods known in the art or any procedure disclosed in the present application. In preferred embodiments, removal of solvent may include, but not limited to: solvent evaporation under atmospheric pressure or reduced pressure/vacuum such as a rotational distillation using Buchi Rotavapor, spray drying, freeze drying, thin film drying, agitated thin film drying, rotary vacuum paddle dryer (RVPD) and the like.

In embodiments, the present application provides amorphous solid dispersion of Pirtobrutinib with a pharmaceutically acceptable carrier, wherein the ratio of Pirtobrutinib and the pharmaceutically acceptable carrier is 1:0.1 to 1:5, w/w.

In other embodiments, the present application provides amorphous solid dispersion of Pirtobrutinib with the pharmaceutically acceptable carrier, wherein the ratio of Pirtobrutinib and the pharmaceutically acceptable carrier is 1:1 to 1:2, w/w.

In a specific embodiment, the present application provides amorphous solid dispersion of Pirtobrutinib with the pharmaceutically acceptable carrier, wherein the ratio of Pirtobrutinib and the pharmaceutically acceptable carrier is 1:1, w/w.

In one specific embodiment, the present application provides an amorphous solid dispersion of Pirtobrutinib with HPMC phthalate (1:1, w/w).

In another specific embodiment, the present application provides an amorphous solid dispersion of Pirtobrutinib with HPC (1:1, w/w).

In yet another specific embodiment, the present application provides an amorphous solid dispersion of Pirtobrutinib with mixture of HPMC phthalate and HPC (1:0.5:0.5, w/w).

In still another specific embodiment, the present application provides an amorphous solid dispersion of Pirtobrutinib with Eudragit L100 (1:1, w/w).

In another specific embodiment, the present application provides an amorphous solid dispersion of Pirtobrutinib with microcrystalline cellulose (MCC) (1:1, w/w).

In yet another specific embodiment, the present application provides an amorphous solid dispersion of Pirtobrutinib with carboxymethyl cellulose (1:1, w/w).

In still another specific embodiment, the present application provides an amorphous solid dispersion of Pirtobrutinib with mannitol (1:1, w/w).

In another specific embodiment, the present application provides an amorphous solid dispersion of Pirtobrutinib with Neusilin (1:1, w/w).

In yet another specific embodiment, the present application provides an amorphous solid dispersion of Pirtobrutinib with cellulose acetate phthalate (1:1, w/w).

In still another specific embodiment, the present application provides an amorphous solid dispersion of Pirtobrutinib with polyvinyl alcohol (1:1, w/w).

In another specific embodiment, the present application provides an amorphous solid dispersion of Pirtobrutinib with polylactic-co-glycolic acid (PLGA) (1:1, w/w).

In still specific embodiment, the present application provides an amorphous solid dispersion of Pirtobrutinib with polyvinyl acetate phthalate (PVAP) (1:1, w/w).

In yet another specific embodiment, the present application provides an amorphous solid dispersion of Pirtobrutinib with sodium alginate (1:1, w/w).

In another specific embodiment, the present application provides an amorphous solid dispersion of Pirtobrutinib with beta cyclodextrin (1:1, w/w).

The amorphous solid dispersion of the present application may be advantageously used in preparation of a pharmaceutical composition comprising Pirtobrutinib with one or more pharmaceutically acceptable excipients.

In another embodiment, the present application provides an amorphous form of Pirtobrutinib. Specifically, the amorphous form of Pirtobrutinib may be characterized by FIG. 2.

In the third aspect, the present application provides a process for the preparation of [(1S)-2,2,2-trifluoro-1-methyl-ethyl]hydrazine or salts of formula (II)

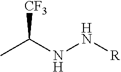

wherein R is a hydrogen or an amine protecting group, comprising asymmetric reductive amination of formula (III) to produce a compound of formula (II)

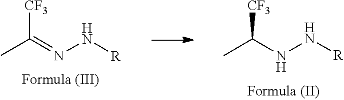

In fourth embodiment, the present application provides a process for the preparation of Pirtobrutinib, comprising,
a) (S)-selective asymmetric reductive amination of formula (III) to produce a compound of formula (II);

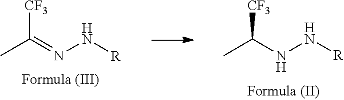

wherein, R is a hydrogen or an amine protecting group
b) converting the compound of formula (II) to Pirtobrutinib of formula (I).

(S)-Selective asymmetric reductive amination is carried out with suitable asymmetric reducing agents. Non limiting example of such (S)-selective asymmetric reductive aminating agents are chiral boranes such as (+)-B-chlorodiisopinochampheylborane (hereinafter referred to as “(+)-DIP-Cl”), (R)-2-methyl-CBS-oxazaborolidine/borane (hereinafter referred to as “(R)—CBS/BH₃”) and the like.

Suitable organic solvent used in the above step include, but are not limited to ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1, 4-dioxane and the like; nitrile solvent, such as acetonitrile, propionitrile, and the like; ester solvents, such as ethyl formate, methyl acetate, ethyl acetate, propyl acetate, butyl acetate, methyl propanoate, ethyl propanoate, methyl butanoate, ethyl butanoate, and the like; alcoholic solvents such as methanol, ethanol, isopropyl alcohol, n-butanol, 1-propanol or the like, ketone solvents, such as acetone, ethyl methyl ketone, diethyl ketone, methyl isobutyl ketone, C3-C6 ketones and the like; aromatic hydrocarbon solvents, such as toluene, xylene, chlorobenzene, tetralin and the like; halogenated hydrocarbons such as dichloromethane, chloroform and the like; aliphatic hydrocarbon solvents, such as n-pentane, n-hexane, n-heptane and the like; mixtures thereof. Specifically, the solvent may be ether solvent.

The reductive amination may be carried out in between about −30° C. and about 30° C., preferably at about −25° C. and about 0° C.

The compound of formula (II) may be converted to Pirtobrutinib of formula (I) by methods disclosed in U.S. Pat. No. 10,342,780 B2 or WO 2022/056100 A1 or any other method known in the art.

The PXRD conditions for the measurement of PXRD peaks of crystalline form and solid dispersions of Pirtobrutinib of the present application are as follows:
Range: 3° 2θ to 40° 2θ in conventional reflection mode
Instrument: PANalytical X-ray Diffractometer
Detector: X'celerator
Source: Copper K-alpha radiation (1.5418 Angstrom).

Differential Scanning Calorimetry (DSC) analysis of crystalline form of Pirtobrutinib of the present application was carried out using a Discovery Series DSC with a ramp of 10° C./minute up to 300° C.

TGA analysis of crystalline form of Pirtobrutinib of the present application was carried out in a Discovery Series instrument with a ramp of 10° C./minute up to 200° C.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### EXAMPLES

### Example-1: Preparation of Crystalline Form P1 of Pirtobrutinib

Pirtobrutinib (100 mg) was dissolved in ethyl acetate (2 mL) at 25-30° C. n-Hexane (1 mL) was added to the obtained solution at 25-30° C. and stirred for 1 hour at same temperature. The precipitated material was filtered and then dried under reduced pressure to afford the title compound.
Yield: 88 mg
PXRD diagram: FIG. 1.

### Example-2: Process for the Preparation of Amorphous Form of Pirtobrutinib

Pirtobrutinib (200 mg) was dissolved in methanol (10 mL) at 25-30° C. The solution was filtered to make it particle free. The obtained clear solution was distilled under reduced pressure at 60° C. to get amorphous form of Pirtobrutinib.

PXRD diagram: FIG. 2

### Example-3: Process for the Preparation of Amorphous Solid Dispersion of Pirtobrutinib with HPMC-P (1:1, w/w)

Pirtobrutinib (200 mg) and HPMC-P (200 mg) were dissolved in methanol (10 ml) at 25-30° C. The obtained solution was filtered to make it particle free and stirred for 15 min to get clear solution. The obtained clear solution was distilled under reduced pressure at 60° C. to get amorphous solid dispersion of Pirtobrutinib with HPMC-P.

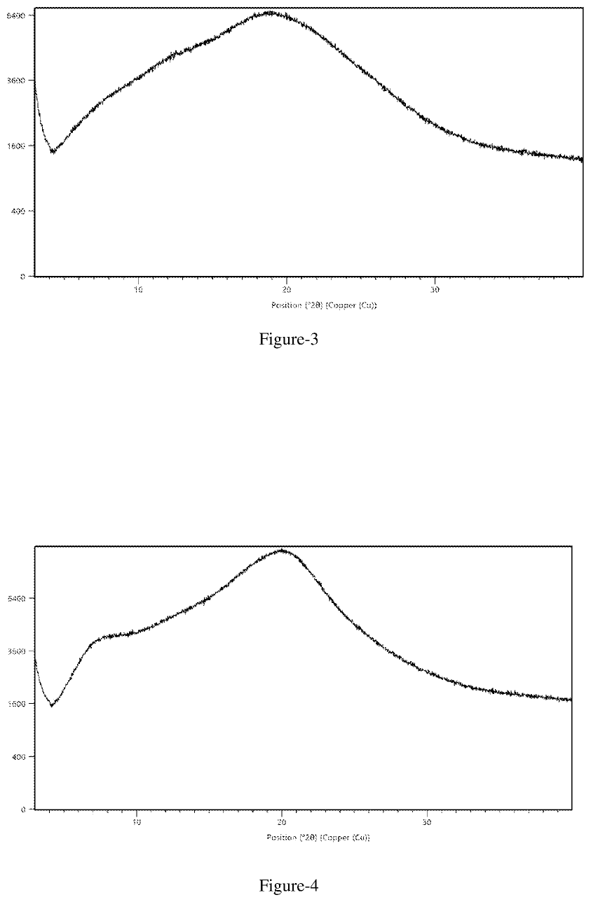

PXRD diagram: FIG. 3

### Example-4: Process for the Preparation of Amorphous Solid Dispersion of Pirtobrutinib with HPC (1:1, w/w)

Pirtobrutinib (200 mg) and HPC (200 mg) were dissolved in methanol (10 ml) at 25-30° C. The solution was filtered to make it particle free and stirred for 15 min to get clear solution. The obtained clear solution was distilled under reduced pressure at 60° C. to get amorphous solid dispersion of Pirtobrutinib with HPC.

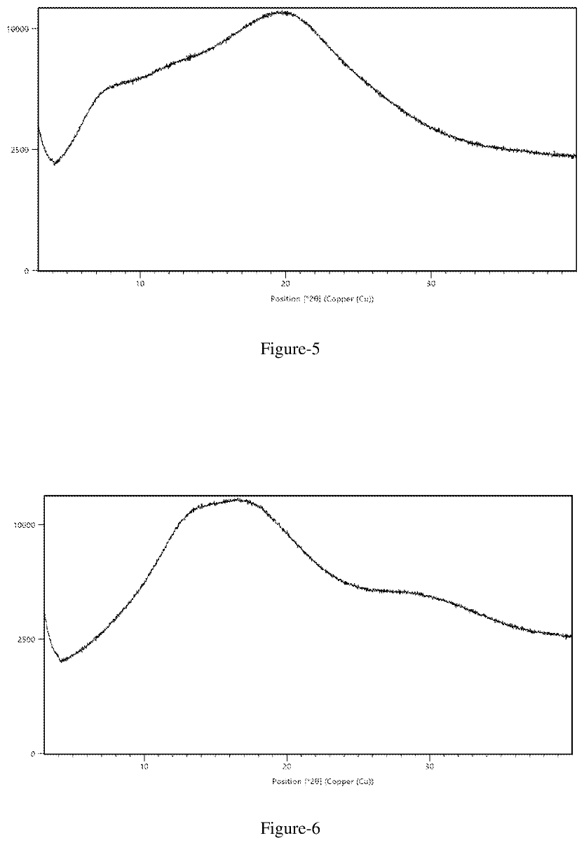

PXRD diagram: FIG. 4

### Example-5: Process for the Preparation of Amorphous Solid Dispersion of Pirtobrutinib with Mixture of HPMC-P and HPC (1:0.5:0.5, w/w)

Pirtobrutinib (200 mg) and mixture of HPMC-P (100 mg) and HPC (100 mg) were dissolved in methanol (10 ml) at 25-30° C. The solution was filtered to make it particle free and stirred for 15 min to get clear solution. The obtained clear solution was distilled under reduced pressure at 60° C. to get amorphous solid dispersion of Pirtobrutinib with HPMC-P and HPC.

PXRD diagram: FIG. 5

### Example-6: Process for the Preparation of Amorphous Solid Dispersion of Pirtobrutinib with Eudragit L100 (1:1, w/w)

Pirtobrutinib (200 mg) and Eudragit L100 (200 mg) were dissolved in methanol (10 mL) at 25-30° C. The solution was filtered to make it particle free. The obtained clear solution was reduced under reduced pressure to obtain amorphous solid dispersion of Pirtobrutinib with Eudragit L100.

PXRD diagram: FIG. 6.

### Example-7: Process for the Preparation of Amorphous Solid Dispersion of Pirtobrutinib with Micro Crystalline Cellulose (MCC)

Pirtobrutinib (1 gr) was dissolved in methanol (20 mL) at 25-30° C. The solution was filtered to make it particle free. Micro crystalline cellulose (1 g) was added to the obtained solution and distilled off the solvent completely under reduced pressure at 60° C. to get the solid compound.

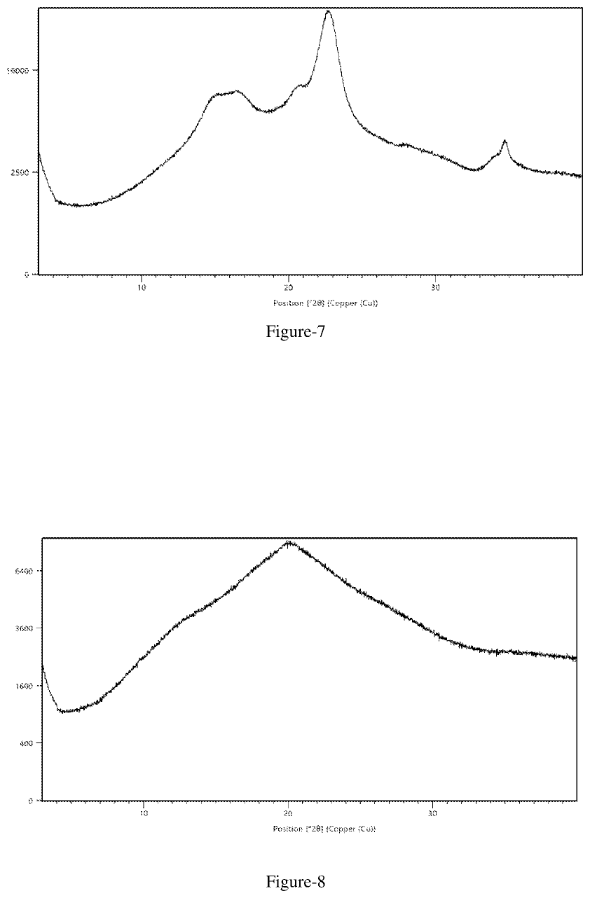

PXRD diagram: FIG. 7

### Example-8: Process for the Preparation of Amorphous Solid Dispersion of Pirtobrutinib with Carboxy Methyl Cellulose (CMC) (1:1, w/w)

Pirtobrutinib (1 g) was dissolved in methanol (20 ml) at 25-30° C. The obtained solution was filtered to make it particle free. Carboxy methyl cellulose (1 g) was added to the obtained solution and distilled off the solvent completely under reduced pressure at 60° C. to get the solid compound.

PXRD diagram: FIG. 8

### Example-9: Process for the Preparation of Amorphous Solid Dispersion of Pirtobrutinib with Mannitol (1:1, w/w)

Pirtobrutinib (1 g) was dissolved in methanol (20 ml) at 25-30° C. The solution was filtered to make it particle free. Mannitol (1 gm) was added to the obtained solution and distilled off the solvent under reduced pressure at 60° C. to get the solid compound.

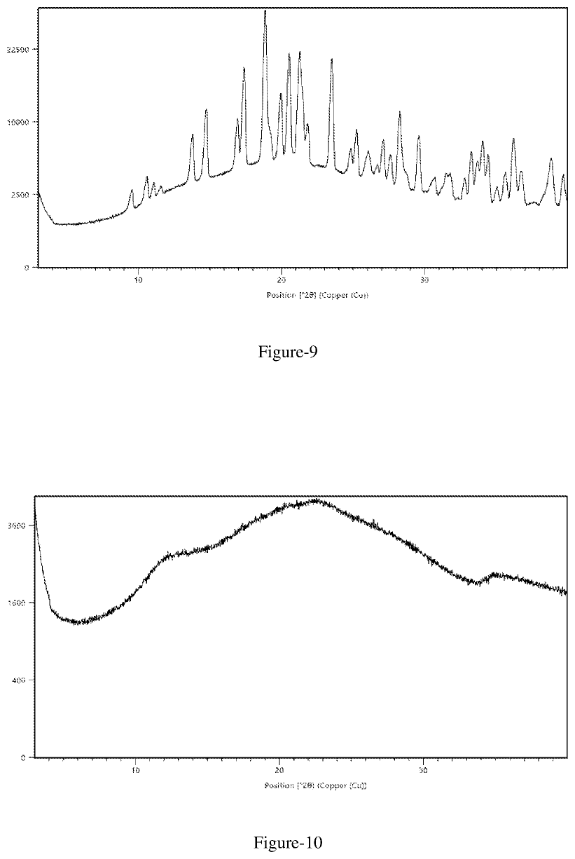

PXRD diagram: FIG. 9

### Example-10: Process for the Preparation of Amorphous Solid Dispersion of Pirtobrutinib with Neusilin (1:1, w/w)

Pirtobrutinib (1 g) was dissolved in methanol (20 ml) at 25-30° C. The solution was filtered to make it particle free. Neusilin (1 gm) was added to the obtained solution and distilled off the solvent under reduced pressure at 60° C. to get the solid compound.

PXRD diagram: FIG. 10

### Example-11: Process for the Preparation of Amorphous Solid Dispersion of Pirtobrutinib with Cellulose Acetate Phthalate (1:1, w/w)

Pirtobrutinib (1 g) was dissolved in methanol (50 mL) at 25-30° C. The solution was filtered to make it particle free. Cellulose acetate phthalate (1 g) was added to the obtained solution and heated to 50-55° C. till dissolution. Distilled off the solvent completely from the solution under reduced pressure to get solid material.

PXRD diagram: FIG. 11.

### Example-12: Process for the Preparation of Amorphous Solid Dispersion of Pirtobrutinib with Polyvinyl Alcohol (1:1, w/w)

Pirtobrutinib (1 g) was dissolved in methanol (30 mL) at 25-30° C. The solution was filtered to make it particle free. Polyvinyl alcohol (1 g) and water (10 ml) were added to the obtained solution and stirred for 5 min to get clear solution. Distilled off the solvent completely from the solution under reduced pressure to get solid material.

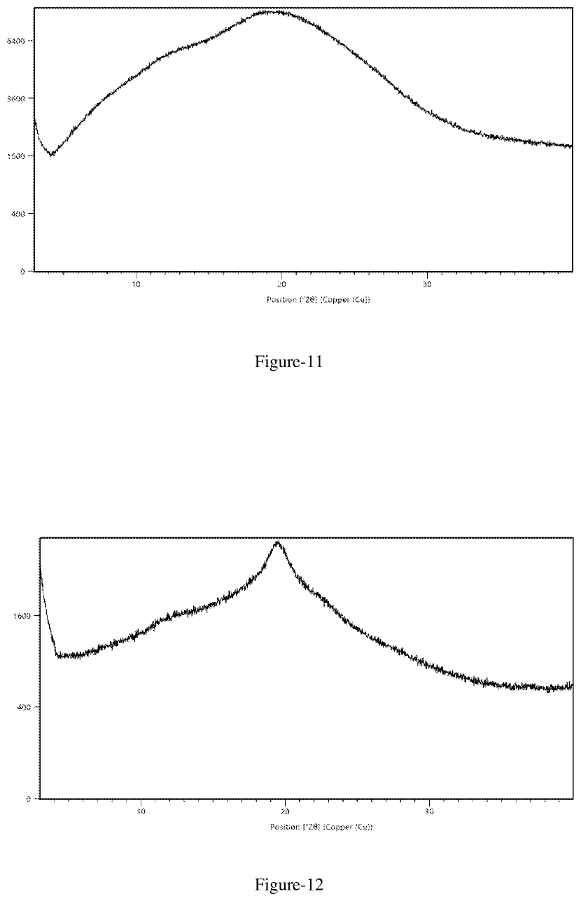

PXRD diagram: FIG. 12.

### Example-13: Process for the Preparation of Amorphous Solid Dispersion of Pirtobrutinib with Polylactic-Co-Glycolic Acid (1:1, w/w)

Pirtobrutinib (1 g) was dissolved in methanol (50 mL) at 25-30° C. The solution was filtered to make it particle free. Polylactic-co-glycolic acid (1 g) and water (10 ml) were added to the above obtained solution and distilled off the solvent completely from the solution under reduced pressure to get solid material.

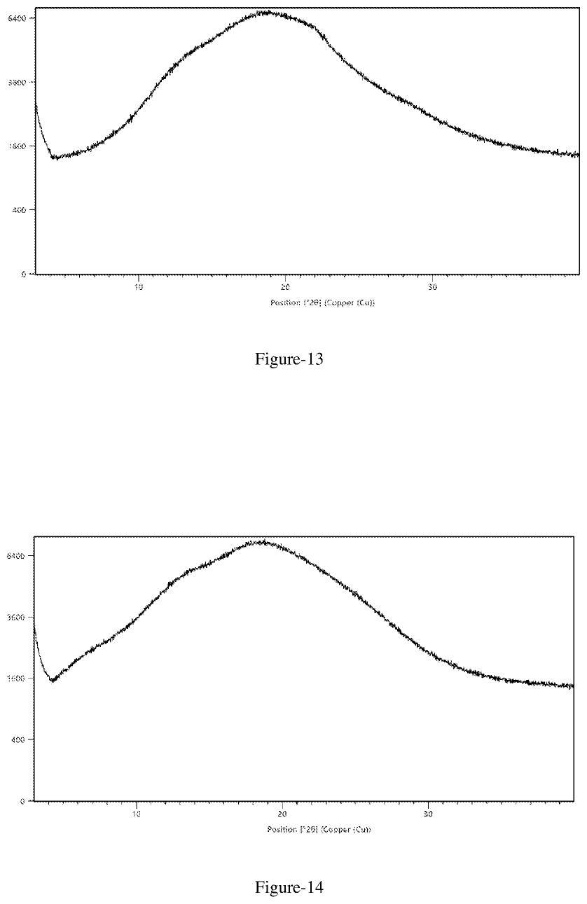

PXRD diagram: FIG. 13.

### Example-14: Process for the Preparation of Amorphous Solid Dispersion of Pirtobrutinib with Polyvinyl Acetate Phthalate (1:1, w/w)

Pirtobrutinib (1 g) was dissolved in methanol (50 mL) at 25-30° C. The solution was filtered to make it particle free. Polyvinyl acetate phthalate (1 g) and water (10 ml) were added to the above solution and stirred for 5 min for dissolution. Distilled off the solvent completely from the solution under reduced pressure to get solid material.

PXRD diagram: FIG. 14.

### Example-15: Process for the Preparation of Amorphous Solid Dispersion of Pirtobrutinib with Sodium Alginate (1:1, w/w)

Pirtobrutinib (1 g) was dissolved in methanol (50 mL) at 25-30° C. The solution was filtered to make it particle free. Sodium alginate (1 g) and water (50 ml) were added to the above solution and stirred for 5 min for dissolution. Distilled off the solvent completely from the solution under reduced pressure to get solid material.

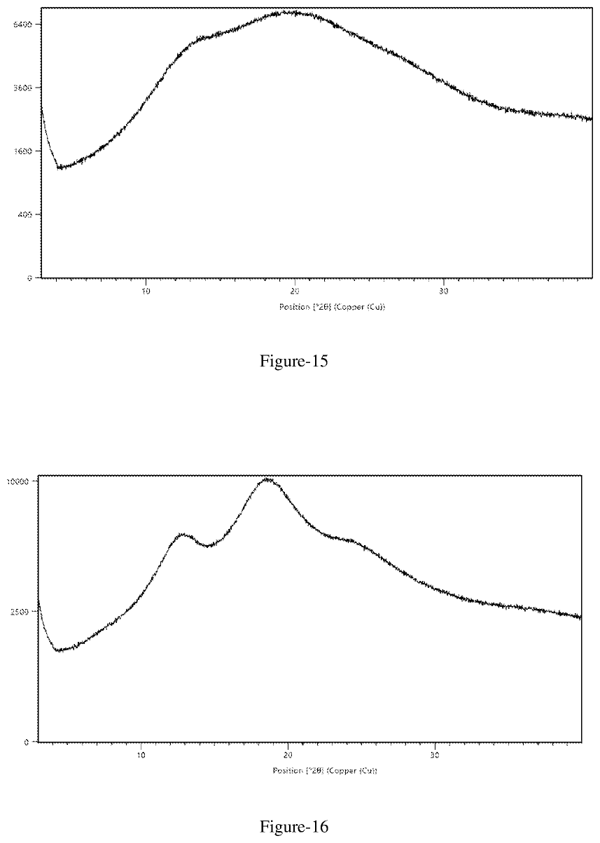

PXRD diagram: FIG. 15.

### Example-16: Process for the Preparation of Amorphous Solid Dispersion of Pirtobrutinib with Beta Cyclodextrin (1:1, w/w)

Pirtobrutinib (4 g) was dissolved in methanol (400 mL) at 25-30° C. The solution was filtered to make it particle free. Beta cyclodextrin (4 g) and water (100 ml) were added to the above solution at 50-55° C. and stirred for 10 min. Distilled off the solvent completely from the solution under reduced pressure to get solid material.

PXRD diagram: FIG. 16.

### Example-17: Process for the Preparation of Amorphous Solid Dispersion of Pirtobrutinib with Polyvinyl Alcohol (1:1, w/w)

Pirtobrutinib (4 g) was dissolved in methanol (200 mL) at 25-30° C. The solution was filtered to make it particle free. Polyvinyl alcohol (4 g) dissolved in water (50 ml) at 50-55° C. and added to the above obtained solution and stirred for 10-15 min at same temperature. Spray dried the obtained solution to get the solid compound.

### Example-18: Process for the Preparation of Amorphous Solid Dispersion of Pirtobrutinib with Polylactic-Co-Glycolic Acid (1:1, w/w)

Pirtobrutinib (4 g) was dissolved in acetone (50 mL) at 25-30° C. The solution was filtered to make it particle free. Polylactic-co-glycolic acid (4 g) was dissolved in tetrahydrofuran (150 ml) at 50-55° C. and added to the above obtained solution and stirred for 10 min. Spray dried the obtained solution to get the solid compound.

### Example-19: Process for the Preparation of Pirtobrutinib Crystalline Form P1

Pirtobrutinib (5 g) was dissolved in acetone (30 mL) at 25-30° C. The solution was filtered to make it particle free. n-Heptane (120 ml) was added to the above particle free solution at 25-30° C. and stirred for 20-24 hours at same temperature. The precipitated solid was filtered and then dried to get the solid material.

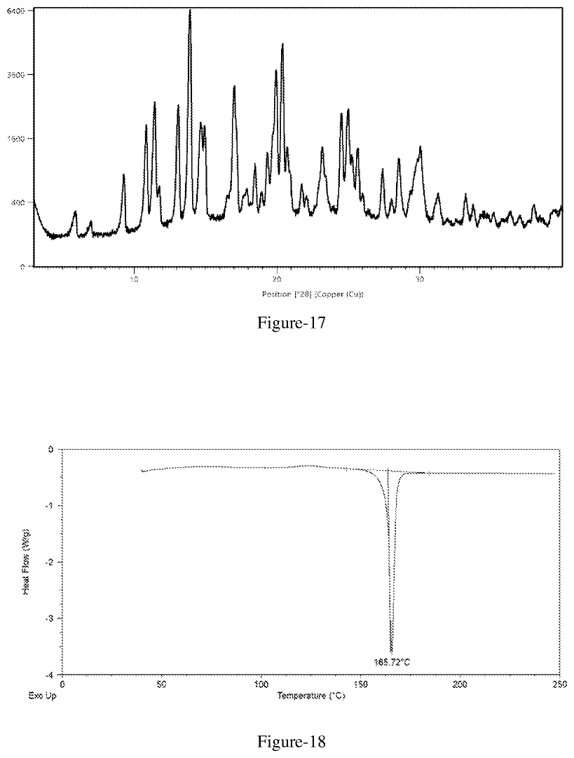

PXRD diagram: Illustrated in FIG. 17.

DSC: Illustrated in FIG. 18.

TGA: Illustrated in FIG. 19.

### Example-20: Process for the Preparation of N′-(1,1,1-Trifluoropropan-2-Ylidene)Benzohydrazide

1,1,1-Trifluoropropan-2-one (18.5 g, 0.165 mol) was added to a suspension of benzohydrazide (15.0 g, 0.110 mol) in toluene (150 mL) at 25-30° C. under nitrogen atmosphere. The reaction mixture was stirred at 110° C. for 16 hours. The reaction mixture was further cooled to 25-30° C. and 1,1,1-trifluoropropan-2-one (9.8 g, 0.088 mol) was added. The obtained reaction mixture was stirred at 110° C. for 6 hours. The reaction mixture was cooled to 25-30° C. and allowed to stand for 1 hour. The resulting suspension was filtered under reduced pressure. The obtained solid was washed with hexane (40 mL) and dried under reduced pressure for 1 hour to obtain N-(1,1,1-trifluoropropan-2-ylidene)benzohydrazide as white solid which was used directly for the next step without any further purification. Yield: 19 g, 76% yield

### Example-21: Process for the preparation of (S)—N′-(1,1,1-trifluoropropan-2-yl)benzohydrazide

(+)-DIP-Cl (50-65% solution in hexane, 15.2 mL, 0.025 moles) was added to a solution of N′-(1,1,1-trifluoropropan-2-ylidene)benzohydrazide (4 g, 0.017 mol) in anhydrous tetrahydrofuran (120 mL) at −25° C. The reaction mixture was stirred at −25° C. for 16 hours. A second lot of (+) DIP-Cl (50-65% solution in hexane, 5.0 mL, 0.0086 moles) was added into the reaction mixture at −25° C. which was then stirred at the same temperature for 6 hours. Thereafter, a third lot of (+) DIP-Cl (50-65% solution in hexane, 2.0 mL, 0.0034 moles) was added into the reaction mixture at −25° C. which was then stirred for 16 hours. Reaction mixture was quenched by addition of methanol (32 mL) into the reaction mixture at −25° C., which was then allowed to warm to 25-30° C. The reaction mixture was concentrated under reduced pressure at 35° C. Ethyl acetate (60 mL), followed by water (40 mL), was charged into the obtained residue. The mixture was stirred for 10 min and the layers were allowed to separate. The organic layer was separated, washed with brine (40 mL), dried over sodium sulfate and concentrated under reduced pressure. The residue was dissolved in dichloromethane (20 mL) and cooled to 0-5° C. n-Hexane (60 mL) was added to the solution at same temperature and stirred for 30 min. The solid was filtered, washed with cold hexane (30 mL) and then dried under reduced pressure to get (S)—N′-(1,1,1-trifluoropropan-2-yl)benzohydrazide (Crop-1, 2.1 g). The filtrate was concentrated, and the obtained residue was dissolved in dichloromethane (10 mL). The solution was cooled to 0-5° C. n-Hexane (30 mL) was added into the solution at 0° C. and then stirred for 30 min. The resulting suspension was filtered. The obtained solid was washed with cold hexane (20 mL) and dried under reduced pressure for 30 min give (S)—N′-(1,1,1-trifluoropropan-2-yl)benzohydrazide (Crop-2, 640 mg).

Analytical data: Mass (m/z): 233.1 [M+H]⁺, Purity by HPLC: 95.15% and Chiral Purity (CHIRALPAK AD-H column): 99.52%

### Example-22: Process for the preparation of (S)—N′-(1,1,1-trifluoropropan-2-yl)benzohydrazide

BH₃-THF complex (1M in THF, 1.6 mL, 0.0016 mol.) was added to (R)-(−)-2-methyl-CBS-oxazaborolidine (361 mg, 0.0013 moles) at 0° C. and the obtained mixture was stirred for 30 min at 0° C. A solution of N′-(1,1,1-trifluoropropan-2 ylidene) benzohydrazide (0.250 g, 0.001 mol.) in anhydrous tetrahydrofuran (8 mL) was added to the above obtained reaction mixture at 0° C. The reaction mixture was allowed to 25-30° C. and stirred at the same temperature for 12-14 hours. The reaction mixture was quenched with methanol (2 mL) and then concentrated under reduced pressure at 40° C. The obtained residue was taken in water (5 mL) and extracted with ethyl acetate (3×15 mL). The ethyl acetate extracts were combined, washed with brine (5 mL), dried over sodium sulfate and concentrated under reduced pressure at 40° C. to obtain the crude product. The crude product was purified by column chromatography (5% ethyl acetate-hexane) to get (S)—N′-(1,1,1-trifluoropropan-2-yl)benzohydrazide (160 mg, 63% yield).

### Example-23: Process for the preparation of [(1S)-2,2,2-trifluoro-1-methyl-ethyl]hydrazine hydrochloride salt

37% Aqueous HCl (4.5 mL, 2.25 vol) solution was added to a solution of (S)—N′-(1,1,1-trifluoropropan-2-yl)benzohydrazide (2 g, 0.0086 moles) in methanol (16 mL) at 0-5° C. and stirred at 90-100° C. for 16-22 hours. The reaction mixture was cooled to 25-30° C. and a second lot of 37% aqueous HCl (1.0 mL, 0.5 vol) was added. The reaction mixture was then stirred at 90-100° C. for 16 hours. The reaction mixture was cooled to 25-30° C. and concentrated under reduced pressure at 40° C. to obtain a solid (~3 g) compound. The obtained solid was suspended in hexane (10 mL) and stirred at 25-30° C. for 30 min. The suspension was filtered, washed with hexane (10 mL) and then dried under reduced pressure to get title compound (900 mg, 64% yield).

Analytical data: Mass (m/z): 129.1 [M+H]⁺ Purity by GC: 99.31%.

### Example 24: Process for the preparation of N′-(1,1,1-trifluoropropan-2-ylidene)benzohydrazide

1,1,1-Trifluoropropan-2-one (12.34 g, 0.11 moles) was added to a suspension of benzohydrazide (10.0 g, 0.073) in ethanol (75 mL) at 25-30° C. under nitrogen atmosphere. The reaction mixture was stirred at 65-70° C. for 8 hours. The reaction mixture was cooled to 25-30° C. and stirred for 18 hours at same temperature. The reaction mixture was further cooled to 0-5° C. and stirred for 2 hours. The resulting suspension was filtered under suction. The obtained solid was washed with hexane (30 mL) and dried under reduced pressure for 1 hour to obtain N′-(1,1,1-trifluoropropan-2-ylidene)benzohydrazide as white solid.

Yield: 16.35 g, 96.74% yield

### Example 25: Process for the preparation of (S)—N′-(1,1,1-trifluoropropan-2-yl)benzohydrazide

(+)-DIP-Cl (50-65% solution in hexane, 255.5 mL, 0.434 moles) was added to a de-gassed suspension of N′-(1,1,1-trifluoropropan-2-ylidene)benzohydrazide (50 g, 0.217 mol) in anhydrous tetrahydrofuran (600 mL) at 5-10° C. The reaction mixture was warmed to 10-15° C. and stirred for 8 hours at the same temperature. The reaction mixture was further warmed to 25-30° C. and stirred for 16 hours at the same temperature. The reaction mixture was cooled to 5-10° C. and while being maintained at that temperature, quenched with methanol (250 mL). The reaction mixture was concentrated under reduced pressure at 40-45° C. Ethyl acetate (500 mL) and water (400 mL) was charged into the obtained residue. The mixture was stirred for 10 min and the layers were allowed to separate. The organic layer was separated, washed with brine (400 mL), dried over sodium sulfate and concentrated under reduced pressure. The residue was dissolved in dichloromethane (250 mL). The obtained solution was cooled to 0-5° C., and while being maintained at that temperature, treated with n-hexane (1000 mL) and then stirred for 2.5 hours. The precipitated solids were filtered, washed with cold hexane (150 mL) and then dried under reduced pressure to get (S)—N′-(1,1,1-trifluoropropan-2-yl)benzohydrazide. Yield: 38.5 g (76.38%) Analytical data: Mass (m/z): 233 [M+H]⁺, Purity by HPLC: 99.8% and Chiral Purity (CHIRALPAK AD-H column): 99.4%

### Example 26: Process for the preparation of [(1S)-2,2,2-trifluoro-1-methyl-ethyl]hydrazine hydrochloride salt

Concentrated HCl (118.5 ml) was added to a solution of (S)—N′-(1,1,1-trifluoropropan-2-yl)benzohydrazide (79 g) in methanol (632 mL) maintained at 0-5° C. The reaction mixture was heated to 65-70° C. and stirred for 7 hours. The reaction mixture was cooled to 0-5° C. and treated with concentrated HCl (118.5 ml). The reaction mixture was heated to 65-70° C. and stirred for 16 hours. The reaction mixture was cooled to 0-5° C. and treated with concentrated HCl (40 ml). The reaction mixture was heated to 65-70° C. and stirred for 24 hours. The reaction mixture was cooled to 45-50° C. and concentrated under reduced pressure at same temperature. Methanol (79 ml) was added to the obtained residue and the resulting mixture was concentrated under reduced pressure. Ethyl acetate (63 ml) was added to the obtained residue and the resulting mixture was concentrated under reduced pressure. The residue was stirred in hexane (395 mL) at 0-5° C. for 60 min to obtain a suspension which was then filtered. The obtained solid was washed with cold hexane, dried at 45-50° C. for 3 hours and then stirred with 5% ethyl acetate-hexane (300 ml) at 25-35° C. for 45 min. The obtained suspension was filtered, washed with 5% ethyl acetate-hexane (32 ml) and then dried under reduced pressure to get title compound (49.6 g, 89.69% yield).

Analytical data: Mass (m/z): 129 [M+H]⁺ Purity by GC: 98.5%.

### Example 27: Preparation of Crystalline Form P1 of Pirtobrutinib

Pirtobrutinib (22 g) was dissolved in ethyl acetate (110 mL) at 25-30° C. n-Hexane (220 mL) was added to the obtained solution at 25-30° C. and stirred for 2-3 h at same temperature. The precipitated material was filtered, washed with n-hexane (110 mL) and then dried under reduced pressure to afford the title compound. Yield: 17.5 g

PXRD diagram: Substantially similar to the PXRD pattern as illustrated in FIG. 1 or FIG. 17.

### Example-28: Stability of Crystalline Form P1 of Pirtobrutinib

Crystalline form P1 of Pirtobrutinib, as prepared in example 27, was kept at 80% RH in open condition and found to be physically and chemically stable. Crystalline form P1 of Pirtobrutinib, as prepared in example 27, was kept under illumination (300-400 nm) for 24 hours and found to be physically and chemically stable.

| | | Exposure at 80% | | Illumination | |
|---|---|---|---|---|---|
| | | RH for 1 day | | (300-400 nm) | |
| 0 Day | | (Open Condition) | | for 24 hours | |
| Purity | PXRD | Purity | PXRD | Purity | PXRD |
| 98.9% | P1 | 98.9% | P1 | 98.9% | P1 |

### Example 29: Stability of Crystalline for P1 of Pirtobrutinib

Crystalline form P1 of Pirtobrutinib, as prepared in example 27, was kept at 25° C./60% RH for 3 months under closed conditions and found to be physically and chemically stable. Similarly, under closed conditions, crystalline form P1 was found to be stable at 40° C./75% RH for 3 months.

| | | 25° C./60% RH | | 40° C./75% RH | |
|---|---|---|---|---|---|
| | | (Closed Condition) | | (Closed Condition) | |
| 0 Day | | (3 months) | | (3 months) | |
| Purity | PXRD | Purity | PXRD | Purity | PXRD |
| 98.9% | P1 | 98.7% | P1 | 98.9% | P1 |

1. Crystalline form P1 of Pirtobrutinib, characterized by data selected from one or more of the following:
a) powdered X-Ray Diffraction (PXRD) pattern having peaks at about 5.80, 6.87, 9.16, 10.74, 11.43, 13.00, 13.78, 14.54±0.2° of 2θ;
b) Differential Scanning Calorimetry (DSC) having endotherm onset at about 163±5° C.

2. A process for preparation of crystalline form P1 of Pirtobrutinib as recited in claim 1, comprising:
a) providing a solution of Pirtobrutinib in a solvent;
b) optionally, heating the mixture obtained in step a);
c) combining the mixture obtained in step-a) or step-b) with anti-solvent;
d) isolating the crystalline form P1 of Pirtobrutinib.

3. The process according to claim 2, wherein the solvent used in step-a) is selected from a group of ethyl acetate and acetone.

4. The process according to claim 2, wherein the anti-solvent used in step-c) is selected from a group of n-hexane and n-heptane.

5. Amorphous solid dispersion comprising Pirtobrutinib and one more pharmaceutically acceptable carrier, wherein the pharmaceutically acceptable carrier is selected from the group consisting of HPMC phthalate, HPC, Eudragit L100, microcrystalline cellulose, carboxymethyl cellulose, mannitol, Neusilin, cellulose acetate phthalate, polyvinyl alcohol, poly lactic-co-glycolic acid, polyvinyl acetate phthalate, sodium alginate, beta cyclodextrin, and mixtures thereof.

6. Amorphous solid dispersion of Pirtobrutinib with pharmaceutically acceptable carrier, according claim 5, wherein the ratio of Pirtobrutinib and pharmaceutically acceptable carrier is 1:0.1 to 1:5, w/w.

7. Amorphous solid dispersion of Pirtobrutinib with pharmaceutically acceptable carrier according claim 5, wherein the ratio of Pirtobrutinib and pharmaceutically acceptable carrier, is 1:1 to 1:2, w/w.

8. A process for the preparation of [(1S)-2,2,2-trifluoro-1-methyl-ethyl]hydrazine or salts of formula (II) 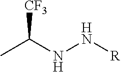
wherein R is a hydrogen or an amine protecting group, comprising asymmetric reductive amination of formula (III) to produce a compound of formula (II)

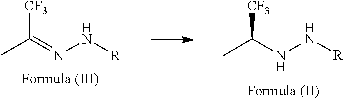

9. A process for the preparation of Pirtobrutinib of formula (I) as recited in claim 2, comprising, a) (S)-selective asymmetric reductive amination of formula (III) to produce a compound of formula (II) 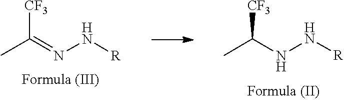
wherein, R is a hydrogen or an amine protecting group; b) converting the compound of formula (II) to Pirtobrutinib of formula (I).

10. The process according to claim 8, wherein (S)-selective asymmetric reductive amination is carried out with suitable asymmetric reductive aminating agent is selected from (+)-B-chlorodiisopinochampheylborane, (R)-2-methyl-CBS-oxazaborolidine/borane.

11. The process according to claim 9, wherein (S)-selective asymmetric reductive amination is carried out with suitable asymmetric reductive aminating agent is selected from (+)-B-chlorodiisopinochampheylborane, (R)-2-methyl-CBS-oxazaborolidine/borane.
